(19) United States
(12) Patent Application Publication
(10) Pub. No.: US 2022/0241247 A1
(43) Pub. Date: Aug. 4, 2022

(54) PHARMACOLOGICAL AGENTS FOR TREATING PROTEIN AGGREGATION DISEASES OF THE EYE

(71) Applicant: PLEX PHARMACEUTICALS, INC., San Diego, CA (US)

(72) Inventors: Sridhar Govinda PRASAD, San Diego, CA (US); Marshall Clarke PETERMAN, Oceanside, CA (US); Santosh SINHA, Ladera Ranch, CA (US)

(21) Appl. No.: 17/615,469

(22) PCT Filed: Jun. 1, 2020
(86) PCT No.: PCT/US20/35592

§ 371 Date: Nov. 30, 2021

Related U.S. Application Data
(60) Provisional application No. 62/855,560, filed on May 31, 2019.

(51) Int. Cl. A61K 31/423; A61K 31/675; A61P 27/12; A61K 9/00

(52) U.S. Cl. CPC A61K 31/423; A61K 9/0048; A61P 27/12; A61K 31/675

(57) ABSTRACT

Methods of treating presbyopia or cataract in a subject in need thereof are provided. The methods require administering to the subject an effective amount of a composition comprising a compound that inhibits the formation of high molecular weight aggregates of human α-A-crystallin. A method of preventing and/or treating transthyretin (TTR)-associated amyloidosis using certain of these compounds is also provided.

### CROSS REFERENCE TO RELATED APPLICATIONS

This application claims the benefit under 35 U.S.C. § 119 of U.S. Provisional Patent Appl. No. 62/855,560 filed on May 31, 2019, and entitled “Pharmacological Agents for Treating Protein Aggregation Diseases of the Eye,” the disclosure of which is incorporated herein by reference in its entirety.

### FIELD OF TECHNOLOGY

Disclosed herein are methods of using 2-(3,5-dichlorophenyl) benzo[d]oxazole-6-carboxylic acid (Tafamidis) and the prodrug of Tafamidis to treat presbyopia or cataract eye diseases.

### BACKGROUND

Cataracts are the leading cause (51%) of blindness worldwide according to the World Health Organization (WHO), particularly in low- and middle-income countries. Data dating back to the beginning of this millennium showed that 30-60% of blindness in Africa and 60-80% in South East Asia is attributable to cataracts. In the United States, the current number of those with cataract is estimated to be more than 25.7 million. Projections from Prevent Blindness research estimate that the number will increase to 38.5 million by 2032, and to 45.6 million by the year 2050. Cataract is a clouding of the eye's lens which blocks or changes the passage of light into the eye. Cataracts usually form in both eyes, but not at the same rate. They can develop slowly or quickly, or progress to a certain point, then not get any worse. Besides aging, other factors may cause cataracts to form. Eye infections, some medicines (such as steroids), injuries or exposure to intense heat or radiation may cause cataracts. Too much exposure to non-visible sunlight (called UV or ultraviolet light) and various diseases, such as diabetes or metabolic disorders, may also contribute to cataracts forming.

The only treatment currently available is surgical extraction of the lens and replacement with an interocular lens which imposes a high burden on public health. Although cataract surgery is generally considered to be safe, there are significant complications: (i) 30-50% of patients in the US having cataract surgery develop opacification of the posterior lens capsule within two years and require laser treatment; (ii) 0.8% have retinal detachments; (iii) 0.6-1.3% are hospitalized for corneal edema or require corneal transplantation and (iv) about 1% are presented with endophthalmitis. In addition, in many remote and poor areas of the developing and under-developed regions of the world, people still remain blind from cataract, primarily due to lack of access to eye care.

Presbyopia is the loss of accommodative ability of the eye resulting in the inability to focus on near objects. Presbyopia affects everyone over the age of 45 and has significant negative impacts on the quality of life. Current treatments for presbyopia include: (i) non-invasive approaches that utilize devices to help improve near and distance vision but do nothing to restore the natural process of accommodation and require constant use of the devices, and (ii) invasive surgical procedures which are associated with major complications including decrease in vision quality, regression effects, anisometropia, corneal ectasia, and haze. Most importantly, none of these methods can reverse presbyopia. Moreover, no treatment option exists that can either prevent or delay the onset of presbyopia.

Stiffening of eye lens and changes in the elasticity of the lens capsule, dimension of eye lens, dimension of the zonular attachment, and ciliary muscle (CM) contractions, have all been proposed as contributing factors for presbyopia. However, human and non-human primate studies suggest that CM function is normal well beyond the onset of presbyopia. By contrast, the human lens increases in stiffness with age in a manner that directly correlates with a loss in accommodative power. The loss in accommodative power can be restored by implanting intraocular lenses made from a flexible polymer suggesting that restoration of lens flexibility is sufficient to restore accommodation. Therefore, a pharmacological agent that could prevent or reverse the hardening of the crystalline lens would provide a promising avenue for a novel non-invasive treatment for presbyopia.

At the molecular level, proteins known as crystallins play a major role in the stiffening of the eye lens. The lens crystallins comprise three isoforms, α, β, and 65 and make up 90% of the eye lens protein content. α crystalline (AC), an ATP-independent chaperone and member of the small heat shock protein (sHsp) family, constitutes 40% of the crystallin protein content. It exists as a hetero-oligomer of two subunits, αA-crystallin (AAC) and αB-crystallin (ABC) and its expression is primarily restricted to the eye lens. It recognizes exposed conformational features in partially unfolded lens proteins and sequesters them from one another, thereby reducing the population of aggregation-prone species that would otherwise lead to various age-related vision impairment.

Multiple studies have established a link between stiffening of the human lens and AC function. Dynamic mechanical analysis measurements have shown that there is a significant increase in the stiffness of the lens with age, particularly in the lens nucleus where a 500- to 1000-fold decrease in elasticity is observed. This increase in lens stiffness correlates with the age-related decline in free AC chaperone concentration as most AC becomes incorporated into high molecular weight (HMW) aggregates by the age of 40-50. This conversion of soluble AC into HMW aggregates is accompanied by a large increase in lens stiffness, presumably because the low level of soluble AC present is not sufficient to chaperone denatured proteins. That age-related decrease in free AC chaperone is responsible for lens stiffness is supported by experiments where human lenses were subjected to heating to mimic the age-related conversion of soluble AC into HMW aggregates and an increase in lens stiffness was observed. Similarly, purified soluble AC forms HMW aggregates when exposed to UV radiation with a loss in chaperone like activity. The HMW aggregate is formed due to the intermolecular cross-linking, particularly S—S bonds, resulting from the oxidation of cysteine sulfhydryl groups (—SH). The formation of this disulfide cross-linked HMW aggregate is thought to be a major contributor in increasing the stiffness and loss of accommodation amplitude of the lens.

It has been suggested that presbyopia is the earliest observable symptom of age-related nuclear (ARN) cataract, a major cause of blindness in the world. The chaperone-like activity (CLA) of AC plays an essential role in maintaining lens transparency. Decreased AC CLA, either as a consequence of AC mutation or age-related modifications, is associated with cataract formation. In congenital cataracts, the most common form of childhood blindness, the majority of cataract-causing mutations have been identified in AC. Some AC mutations directly result in reduced AC solubility and decreased chaperone activity and AC knockout studies conducted in mice resulted in early onset of cataracts. The concept that AC CLA can be modulated through allosteric mechanisms was initially derived from studies which found that cations or small molecules can increase or decrease CLA in AC using in vitro chaperone assays. Thus, pharmacological modulation of AC CLA is a plausible approach for cataract treatment and/or prevention.

Given the need for noninvasive treatment that can protect and restore the accommodative ability of the eye lost in presbyopia and given that formation of HMW AC aggregates is a major causative factor underlying presbyopia, there is a need for the development of pharmacological agents that can selectively delay and/or reverse the HMW AC aggregate formation.

### SUMMARY

Tafamidis (CAP4349; see structure below), is an FDA approved drug used for the treatment of transthyretin mediated cardiomyopathy (ATT-CM) (Falk RH, 2019, Eur Heart J. 40(12):1009-1012). Transthyretin amyloid cardiomyopathy is caused by the deposition of transthyretin amyloid fibrils in the myocardium. The deposition occurs when wild-type or variant transthyretin becomes unstable and misfolds. Tafamidis binds to transthyretin, preventing tetramer dissociation and amyloidogenesis.

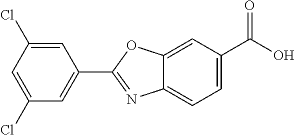 [SMILES: O=C(O)c1ccc2nc(-c3cc(Cl)cc(Cl)c3)oc2c1]

As described herein, the present inventors found that CAP4349 is also able to prevent aggregation of human ACC as well as dissolve ACC inclusions as described below. Also described herein are prodrugs of CAP4349. Prodrugs are molecules with little or no pharmacological activity that are converted to the active parent drug in vivo by enzymatic or chemical reactions or by a combination of the two.

Accordingly, the present disclosure provides a method for treating presbyopia or cataract in a subject. The method comprises administering to the subject a pharmaceutical composition comprising a therapeutically effective amount a compound having formula (I)

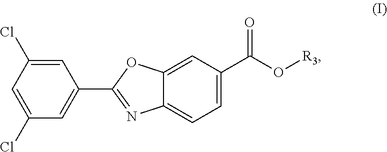

or a solvate or a pharmaceutically acceptable salt thereof, wherein R₃ is as defined herein below.

The present disclosure also provides a method for preventing and/or treating transthyretin (TTR)-associated amyloidosis using a pharmaceutical composition comprising a therapeutically effective amount a compound having formula (I) or a solvate or a pharmaceutically acceptable salt thereof, wherein R₃ is as defined herein below.

### BRIEF DESCRIPTION OF THE DRAWINGS

FIG. 1A is a SDS-PAGE gel showing inhibition of UV induced aggregation of human ACC by the example compound CAP4349 (Tafamidis).

FIG. 1B is a graph showing inhibition of UV induced aggregation of human ACC by the example compound CAP4349 (Tafamidis).

FIG. 2A is a set of kinetic curves showing delayed aggregation of lysozyme by AAC in presence of CAP4349-M in a dose dependent manner.

FIG. 2B is a bar graph showing cumulative decreased aggregation of lysozyme by AAC in the presence of different concentrations of CAP4349-M, measured at 43 minutes from the initiation of aggregation. The bars show mean relative absorbance±SD (n=3 or 4).

FIG. 3A is a graph showing protection from heat induced cell death of human lens epithelial cells conferred by the example compound CAP4349.

FIG. 3B is a graph showing protection from UV induced cell death of human lens epithelial cells conferred by the example compound CAP4349.

FIG. 4A is a set of immunofluorescence image showing dissolution of aggregated green fluorescence protein (GFP)-tagged ACC mutant (R116C) protein upon treatment with the example compound CAP4349. FIG. 3A also shows localization of autophagy inducing ubiquitin-binding protein p62 with aggregated mutant ACC using P62/SQSTM1 antibody for staining.

FIG. 4B is a bar graph showing the effect of increasing amounts of the example compound CAP4349 in dissolving inclusions of ACC and p62 as measured by GFP fluorescence.

FIG. 5A is a bright field image and a graph showing the ability of the example compound CAP4349 to prevent the formation of high molecular weight aggregates of bovine eye lens protein.

FIG. 5A is a bright field image showing the ability of the example compound CAP4349 to prevent the formation of high molecular weight aggregates of human eye lens protein.

FIG. 7 is a set of photographs and a graph. The photographs show porcine lenses pretreated with 125 μM CAP4349 (or vehicle) and irradiated or not irradiated with UV light. Representative dark field and bright field images are shown. The graph shows median pixel intensities ±SD and p value (t test) from dark field images.

### DETAILED DESCRIPTION

The present disclosure describes the ability of CAP4349 (Tafamidis) to prevent aggregation of human ACC as well as dissolve ACC inclusions. Tafamidis is a FDA approved drug for the treatment of the heart disease (cardiomyopathy) caused by transthyretin mediated amyloidosis (ATTR-CM) in adults. Similar to cataracts, ATTR-CM is also a slow progressive condition characterized by the buildup of abnormal deposits of specific proteins called amyloids in the body's organs and tissues interfering with their normal functioning. Pharmacologically, Tafamidis acts like a chaperone and stabilizes the correctly folded tetrameric form of transthyretin (TTR), thereby inhibiting its dissociation. In people suffering form ATTR-CM, the individual monomers of transthyretin fall away from the tetramer, misfold, and form aggregates.

As described herein, CAP4349 was found to be able to also prevent aggregation of human ACC as well as dissolve ACC inclusions. Based on this observation, disclosed herein is a method for treating presbyopia or cataract by administering to a subject CAP4349 or a prodrug thereof. Prodrugs are inactive compounds created by chemical modification of biologically active compounds. Prodrugs are most commonly used to increase permeability of compounds by masking the polar functional groups and hydrogen bonds with ester or amide linkers to increase lipophilicity. As noted above, prodrugs are converted to the active parent drug in vivo by enzymatic or chemical reactions or by a combination of the two.

More specifically, in a first aspect of the present technology, a method for treating presbyopia or cataract in a subject in need thereof is provided. The method comprises administering to the subject a pharmaceutical composition comprising a therapeutically effective amount a compound having the formula (I)

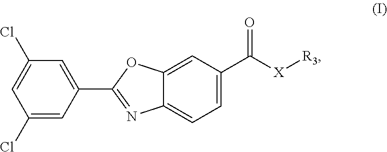

or a solvate or a pharmaceutically acceptable salt thereof,
wherein

R₃ is selected from the group consisting of hydrogen, an amino-acid, C₁₋₁₀alkyl, C₁₋₁₀ branched-alkyl, C₁₋₁₀ hydroxyalkyl, C₁₋₁₀ haloalkyl, C₂₋₆ alkenyl, C₂-C₆ alkylaryl, C₁₋₆ alkyl (C₃-C₆) C₆) cycloalkyl, C₁₋₆ alkylNH, NC₁₋₆ dialkylamine, C₁₋₆, alkyl-pyrrolidine, C₁-C₆ alkyl-piperidine, C₁₋₆ alkyl morpholine, pthalidyl,

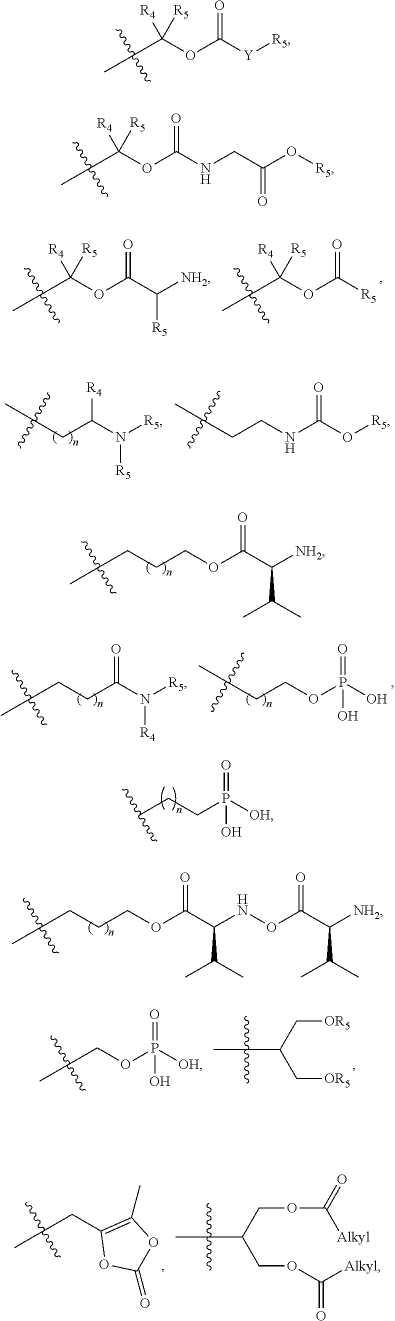

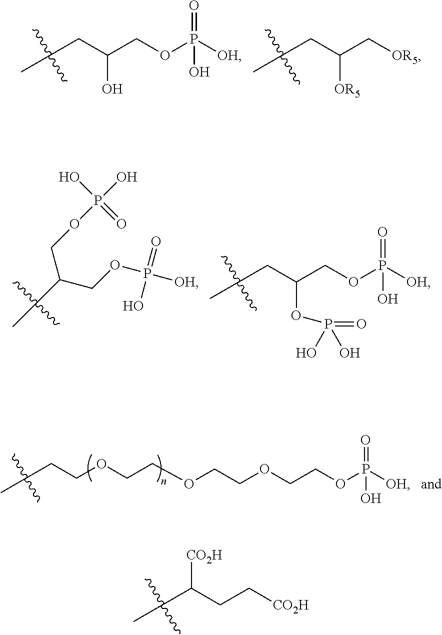

wherein n is a number between 0 and 6;

R₄ and R₅ are independently selected from hydrogen, C₁₋₆ alkyl, C₁₋₆ haloalkyl, C₁₋₆ hydroxyalkyl, C₂₋₆ alkoxyalkyl, aralkyl, C₂₋₆ alkenyl, C₂₋₆ alkynyl, 3 to 6 membered cycloalkyl optionally substituted with at least one group selected from W, 4 to 6 membered heterocyclyl optionally substituted with at least one group selected from W,

wherein W is selected from C₁₋₆ alkyl, C₂₋₆ alkenyl, C₂₋₆ alkynyl, halo, NH₂, C₁₋₆ alkylNH, NC₁₋₆ dialkylamine, C₁₋₆ alkoxy, and hydroxyl; and Y is O, S, or NR₆, wherein R₆ is hydrogen or C₁₋₆ alkyl; and

wherein X is O or NR₆, wherein R₆ is hydrogen or C₁₋₆ alkyl.

The disclosure also provides for prodrugs comprising compounds having formula (I) and methods of preventing and/or treating transthyretin (TTR)-associated amyloidosis.

Accordingly, in a second aspect of the present technology, provided herein is a method for treating transthyretin (TTR)-associated amyloidosis in a subject in need thereof. The method comprises administering to the subject a pharmaceutical composition comprising a therapeutically effective amount a compound having formula (I)

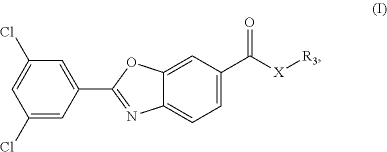

or a solvate or a pharmaceutically acceptable salt thereof,
wherein

R₃ is selected from the group consisting of an amino-acid, C₁₋₁₀ alkyl, C₁₋₁₀ branched-alkyl, C₁₋₁₀ hydroxyalkyl, C₁₋₁₀ haloalkyl, C₂₋₆ alkenyl, C₁₋₆ alkylaryl, C₁-C₆ alkyl (C₃-C₆) cycloalkyl, C₁₋₆ alkylNH, NC₁₋₆ dialkylamine, C₁₋₆, alkyl-pyrrolidine, C₁-C₆ alkyl-piperidine, C₁₋₆ alkyl morpholine, pthalidyl,

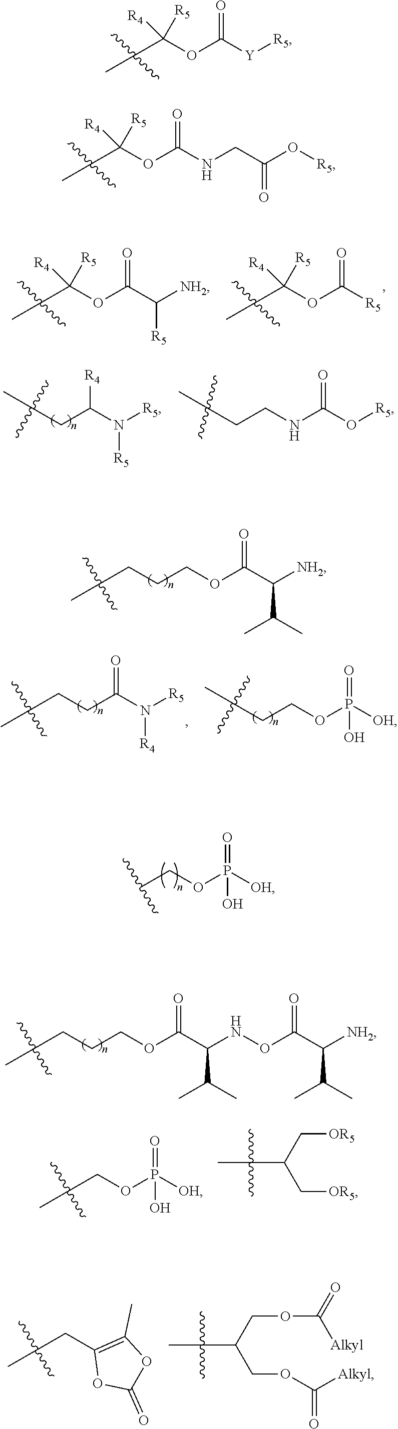

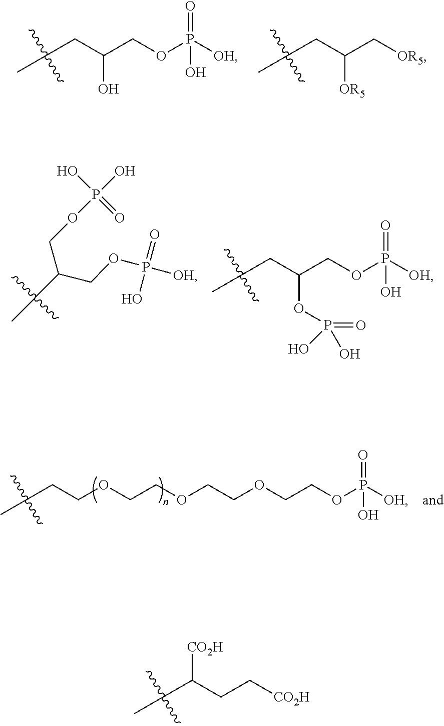

wherein n is a number between 0 and 6;

R₄ and R₅ are independently selected from hydrogen, C₁₋₆ alkyl, C₁₋₆ haloalkyl, C₁₋₆ hydroxyalkyl, C₂₋₆ alkoxyalkyl, aralkyl, C₂₋₆ alkenyl, C₂₋₆ alkynyl, 3 to 6 membered cycloalkyl optionally substituted with at least one group selected from W, 4 to 6 membered heterocyclyl optionally substituted with at least one group selected from W,

wherein W is selected from C₁₋₆ alkyl, C₂₋₆ alkenyl, C₂₋₆ alkynyl, halo, NH₂, C₁₋₆ alkylNH, NC₁₋₆ dialkylamine, C₁₋₆ alkoxy, and hydroxyl; and Y is O, S, or NR₆, wherein R₆ is hydrogen or C₁₋₆ alkyl; and

wherein X is O or NR₆, wherein R₆ is hydrogen or C₁₋₆ alkyl.

### Compounds and Definitions

The terms “halo” and “halogen” as used herein refer to an atom selected from fluorine (fluoro, —F), chlorine (chloro, —Cl), bromine (bromo, —Br), and iodine (iodo, —I).

The term “alkyl”, used alone or as a part of a larger moiety such as e.g., “haloalkyl”, means a saturated monovalent straight or branched hydrocarbon radical having, unless otherwise specified, 1-10 carbon atoms and includes, for example, methyl, ethyl, n-propyl, isopropyl, n-butyl, sec-butyl, isobutyl, tert-butyl, n-pentyl, n-hexyl, n-heptyl, n-octyl, n-nonyl, n-decyl and the like. “Monovalent” means attached to the rest of the molecule at one point.

The terms “cycloalkyl” used alone or as part of a larger moiety, refers to a saturated cyclic aliphatic monocyclic, bicyclic or tricyclic ring system, as described herein, having from, unless otherwise specified, 3 to 10 carbon ring atoms. Monocyclic cycloalkyl groups include, without limitation, cyclopropyl, cyclobutyl, cyclopentyl, cyclopentenyl, cyclohexyl, cyclohexenyl, cycloheptyl, cycloheptenyl, and cyclooctyl. Bicyclic cycloalkyl groups include e.g., cycloalkyl group fused to another cycloalkyl group, such as decalin or a cycloalkyl group fused to an aryl group (e.g., phenyl) or heteroaryl group, such as tetrahydronaphthalenyl, indanyl, 5,6,7,8-tetrahydroquinoline, and 5,6,7,8-tetrahydroisoquinoline. An example of a tricyclic ring system is adamantane. It will be understood that the point of attachment for bicyclic cycloalkyl groups can be either on the cycloalkyl portion or on the aryl group (e.g., phenyl) or heteroaryl group that results in a stable structure. It will be further understood that when specified, optional substituents on a cycloalkyl may be present on any substitutable position and, include, e.g., the position at which the cycloalkyl is attached.

The term “heterocyclyl” means a 4-, 5-, 6- and 7-membered saturated or partially unsaturated heterocyclic ring containing 1 to 4 heteroatoms independently selected from N, O, and S. The terms “heterocycle”, “heterocyclyl”, “heterocyclyl ring”, “heterocyclic group”, “heterocyclic moiety”, and “heterocyclic radical”, may be used interchangeably. A heterocyclyl ring can be attached to its pendant group at any heteroatom or carbon atom that results in a stable structure. Examples of such saturated or partially unsaturated heterocyclic radicals include, without limitation, tetrahydrofuranyl, tetrahydrothienyl, terahydropyranyl, pyrrolidinyl, pyrrolidonyl, piperidinyl, oxetanyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, and tetrahydropyrimidinyl. A heterocyclyl group may be mono or bicyclic. Unless otherwise specified, bicyclic heterocyclyl groups include, e.g., unsaturated or saturated heterocyclic radicals fused to another unsaturated heterocyclic radical or aromatic or heteroaryl ring, such as for example, chromanyl, 2,3-dihydrobenzo[b][1,4]dioxinyl, tetrahydronaphthyridinyl, indolinonyl, dihydropyrrolotriazolyl, imidazopyrimidinyl, quinolinonyl, dioxaspirodecanyl. It will be understood that the point of attachment for bicyclic heterocyclyl groups can be on the heterocyclyl group or aromatic ring that results in a stable structure. It will also be understood that when specified, optional substituents on a heterocyclyl group may be present on any substitutable position and, include, e.g., the position at which the heterocyclyl is attached.

As used herein, the term “aryl”, used alone or in conjunction with other terms, refers to a 6-14 membered aromatic ring containing only ring carbon atoms. The aryl ring may be monocyclic, bicyclic, or tricyclic. Non-limiting examples include phenyl, naphthyl, biphenyl, anthracenyl, and the like. It will also be understood that when specified, five optional substituents on an aryl group may be present on any substitutable position. In an embodiment, the aryl group is unsubstituted or mono- or di-substituted.

The term “heteroaryl” used alone or as part of a larger moiety as in “heteroarylalkyl”, “heteroarylalkoxy”, or “heteroarylaminoalkyl”, refers to a 5-10 -membered aromatic radical containing 1-4 heteroatoms selected from N, O, and S and includes, for example, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The term “heteroaryl” may be used interchangeably with the terms “heteroaryl ring”, “heteroaryl group”, or “heteroaromatic”. The terms “heteroaryl and “heteroar-”, as used herein, also include groups in which a heteroaromatic ring is fused to one or more aryl rings, where the radical or point of attachment is on the heteroaromatic ring. Nonlimiting examples include indolyl, indazolyl, benzimidazolyl, benzthiazolyl, quinolyl, quinazolinyl, and quinoxalinyl. A heteroaryl group may be mono- or bicyclic. It will be understood that when specified, optional substituents on a heteroaryl group may be present on any substitutable position and, include, e.g., the position at which the heteroaryl is attached.

As used herein the terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

As used herein, the term “therapeutically effective amount” means the amount of the pharmaceutical composition that will elicit the biological or medical response of a subject in need thereof that is being sought by the researcher, veterinarian, medical doctor or other clinician. In some embodiments, the subject in need thereof is a mammal. In some embodiments, the mammal is human.

All stereoisomers of the present compounds (for example, those which may exist due to asymmetric carbons on various substituents), including enantiomeric forms and diastereomeric forms, are contemplated within the scope of this technology. Individual stereoisomers of the compounds of the technology may, for example, be substantially free of other isomers (e.g., as a pure or substantially pure optical isomer having a specified activity), or may be admixed, for example, as racemates or with all other, or other selected, stereoisomers. The chiral centers of the present technology may have the S or R configuration as defined by the International Union of Pure and Applied Chemistry (IUPAC) 1974 Recommendations. The racemic forms can be resolved by physical methods, such as, for example, fractional crystallization, separation or crystallization of diastereomeric derivatives or separation by chiral column chromatography. The individual optical isomers can be obtained from the racemates by any suitable method, including without limitation, conventional methods, such as, for example, salt formation with an optically active acid followed by crystallization.

If, for instance, a particular enantiomer of a compound of the present technology is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be appreciated that the compounds, as described herein, may be substituted with any number of substituents or functional moieties. In general, the term “substituted” whether preceded by the term “optionally” or not, and substituents contained in formulas of this technology, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this technology, heteroatoms such as nitrogen may have hydrogen substituents and/or any permissible substituents of organic compounds described herein which satisfy the valencies of the heteroatoms. Furthermore, this technology is not intended to be limited in any manner by the permissible substituents of organic compounds. Combinations of substituents and variables envisioned by this technology are preferably those that result in the formation of stable compounds useful in the treatment, for example, of neurodegenerative disorders. The term “stable,” as used herein, preferably refers to compounds which possess stability sufficient to allow manufacture and which maintain the integrity of the compound for a sufficient period of time to be detected and preferably for a sufficient period of time to be useful for the purposes detailed herein.

### Prodrugs

Prodrugs are pharmacologically inactive medications that have to be converted to an active form through chemical reactions, such as hydrolysis or phosphorylation. Prodrug strategies are most commonly used to increase permeability of compounds by masking the polar functional groups and hydrogen bonds with ester or amide moieties to increase lipophilicity. Both permeability by passive diffusion and the transporter-mediated process have been modulated by prodrug approaches. In the case of a drug having a carboxyl group as in tafamidis (CAP4349), a prodrug can be obtained by conversion of the carboxyl group to an ester or an amide. For carboxyl group containing drugs, simple alkyl esters may be preferred for increasing passive diffusion permeability. Ethyl ester is the most common prodrug of this type. Other promoieties include aryl, double esters with diols, cyclic carbonates, and lactones. All of these promoieties are contemplated herein as prodrugs of tafamidis (CAP4349). Double esters are prepared to increase the recognition by esterases through the second ester. Cyclic carbonate prodrugs (e.g., lenampicillin) are designed to be labile in plasma to avoid nonproductive metabolism by cellular esterases. Prodrugs that hydrolyze in blood or plasma by blood-borne enzymes are beneficial, to increase oral bioavailability and systemic circulation of the active principle. Double esters and cyclic carbonate prodrugs are designed for this purpose. Lactone prodrugs are developed for specific targeting.

### Definition of Exemplary Compounds

In one embodiment, in each of the two aspects, R₃ is an amino acid.

In one embodiment, in each of the two aspects, R₄ is hydrogen and R₅ is a methyl or ethyl.

In one embodiment, in each of the two aspects, R₃ is a C₁₋₆ alkyl.

In one embodiment, in each of the two aspects, X is N.

In one embodiment, in each of the two aspects, X is O.

In one embodiment, in each of the two aspects, R₃ is

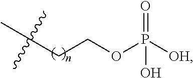

wherein n is 0.

In one embodiment, in each of the two aspects, R₃ is

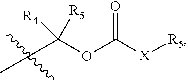

X is O, R₄ is H and R₅ is CH₃.

In one embodiment, in each of the two aspects, R₃ is

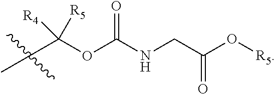

In one embodiment, in each of the two aspects, the compound is

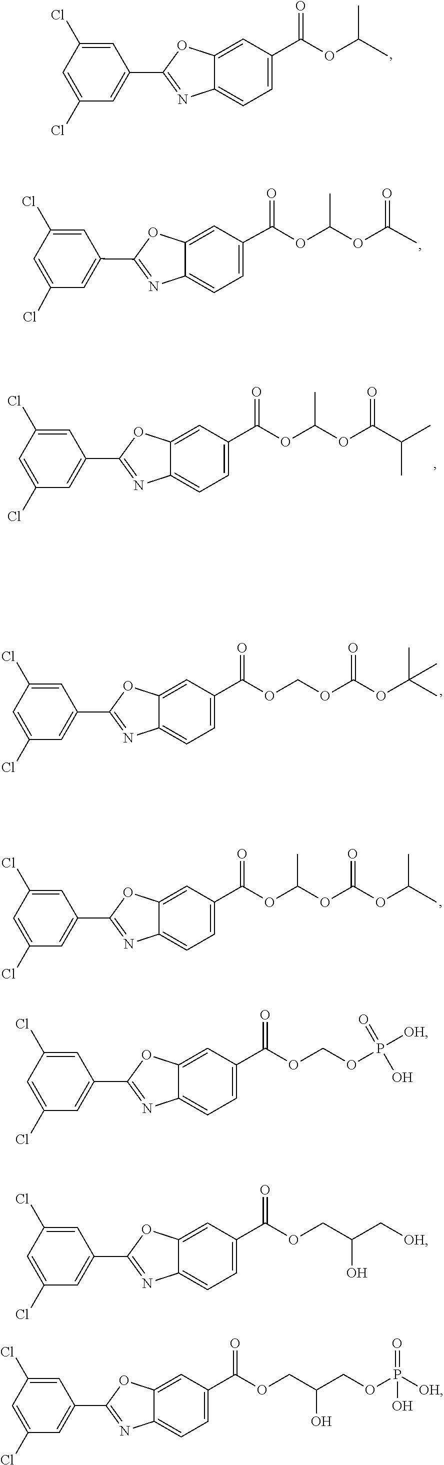

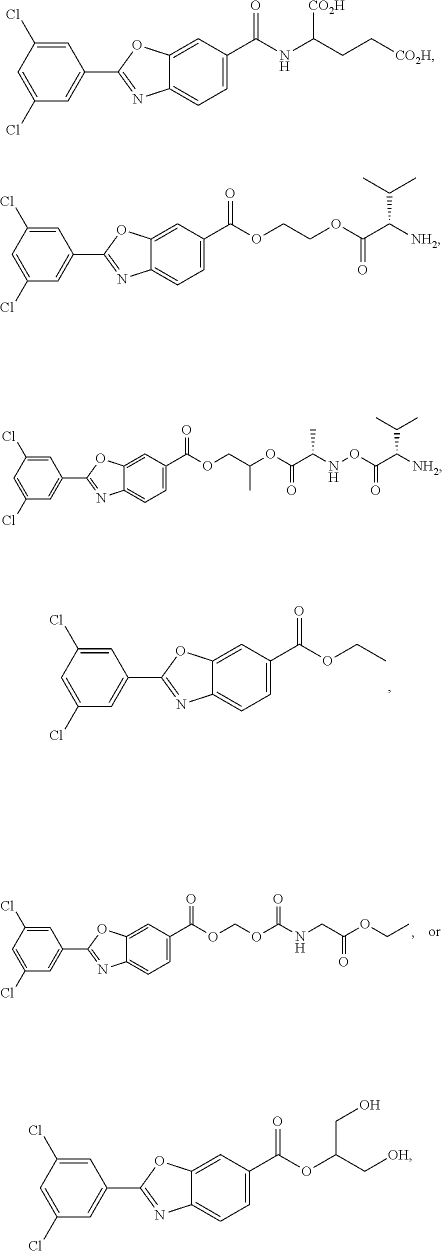 [SMILES: O=C(O)CCC(NC(=O)c1ccc2nc(-c3cc(Cl)cc(Cl)c3)oc2c1)C(=O)O ; CC(C)[C@H](N)C(=O)OCCOC(=O)c1ccc2nc(-c3cc(Cl)cc(Cl)c3)oc2c1 ; CC(COC(=O)c1ccc2nc(-c3cc(Cl)cc(Cl)c3)oc2c1)OC(=O)[C@H](C)NOC(=O)[C@@H](N)C(C)C ; CCOC(=O)c1ccc2nc(-c3cc(Cl)cc(Cl)c3)oc2c1 ; CCOC(=O)CNC(=O)OCOC(=O)c1ccc2nc(-c3cc(Cl)cc(Cl)c3)oc2c1 ; O=C(OC(CO)CO)c1ccc2nc(-c3cc(Cl)cc(Cl)c3)oc2c1]

or a solvate or a pharmaceutically acceptable salt thereof.

In one embodiment, in each of the two aspects, the subject is a human.

In one embodiment, in each of the two aspects, the pharmaceutical composition is formulated for topical ophthalmic administration.

In all of the compounds described herein that include substituent alternatives that may be substituted, such as, for R₄ and R₅, the substitutions are typically, independently of one another, selected from amongst the groups described in connection with structural formula (I).

Those of skill in the art will appreciate that many of the prodrugs described herein, may exhibit the phenomena of tautomerism, conformational isomerism, geometric isomerism and/or optical isomerism. For example, the prodrugs may include one or more chiral centers and/or double bonds and as a consequence may exist as stereoisomers, such as double-bond isomers (i.e., geometric isomers), enantiomers and diasteromers and mixtures thereof, such as racemic mixtures. As another example, the prodrugs may exist in several tautomeric forms, including the enol form, the keto form and mixtures thereof. It should be understood that the present technology encompasses any tautomeric, conformational isomeric, optical isomeric and/or geometric isomeric forms of the prodrugs, as well as mixtures of these various different isomeric forms.

### Pharmaceutical Compositions

Depending upon the nature of the various substituents, the prodrugs described herein may be in the form of salts. Such salts include salts suitable for pharmaceutical uses (“pharmaceutically-acceptable salts”), salts suitable for veterinary uses, etc. Such salts may be derived from acids or bases, as is well-known in the art.

In one embodiment, the salt is a pharmaceutically acceptable salt. Generally, pharmaceutically acceptable salts are those salts that retain substantially one or more of the desired pharmacological activities of the parent compound and which are suitable for administration to humans. Pharmaceutically acceptable salts include acid addition salts formed with inorganic acids or organic acids. Inorganic acids suitable for forming pharmaceutically acceptable acid addition salts include, by way of example and not limitation, hydrohalide acids (e.g., hydrochloric acid, hydrobromic acid, hydriodic, etc.), sulfuric acid, nitric acid, phosphoric acid, and the like. Organic acids suitable for forming pharmaceutically acceptable acid addition salts include, by way of example and not limitation, acetic acid, trifluoroacetic acid, propionic acid, hexanoic acid, cyclopentanepropionic acid, glycolic acid, oxalic acid, pyruvic acid, lactic acid, malonic acid, succinic acid, malic acid, maleic acid, fumaric acid, tartaric acid, citric acid, palmitic acid, benzoic acid, 3-(4-hydroxybenzoyl) benzoic acid, cinnamic acid, mandelic acid, alkylsulfonic acids (e.g., methanesulfonic acid, ethanesulfonic acid, 1,2-ethane-disulfonic acid, 2-hydroxyethanesulfonic acid, etc.), arylsulfonic acids (e.g., benzenesulfonic acid, 4-chlorobenzenesulfonic acid, 2-naphthalenesulfonic acid, 4-toluenesulfonic acid, camphorsulfonic acid, etc.), 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

Pharmaceutically acceptable salts also include salts formed when an acidic proton present in the parent compound is either replaced by a metal ion (e.g., an alkali metal ion, an alkaline earth metal ion or an aluminum ion) or coordinates with an organic base (e.g., ethanolamine, diethanolamine, triethanolamine, N-methylglucamine, morpholine, piperidine, dimethylamine, diethylamine, etc.).

The prodrugs described herein, as well as the salts thereof, may also be in the form of hydrates, solvates and N-oxides, as are well-known in the art. Unless specifically indicated otherwise, the expression “prodrug” is intended to encompass such salts, hydrates, solvates and/or N-oxides. Specific exemplary salts include, but are not limited to, mono- and di-sodium salts, mono- and di-potassium salts, mono- and di-lithium salts, mono- and di-alkylamino salts, mono-magnesium salts, mono-calcium salts and ammonium salts.

The actual amount of the compound to be administered in any given case will be determined by a physician taking into account the relevant circumstances, such as the severity of the condition, the age and weight of the patient, the patient's general physical condition, the cause of the condition, and the route of administration.

The patient will be administered the compound orally in any acceptable form, such as a tablet, liquid, capsule, powder and the like, or other routes may be desirable or necessary, particularly if the patient suffers from nausea. Such other routes may include, without exception, transdermal, parenteral, subcutaneous, intranasal, via an implant stent, intrathecal, intravitreal, topical to the eye, back to the eye, intramuscular, intravenous, and intrarectal modes of delivery. Additionally, the formulations may be designed to delay release of the active compound over a given period of time, or to carefully control the amount of drug released at a given time during the course of therapy.

In certain embodiments, there are provided pharmaceutical compositions including at least one compound having formula (I) in a pharmaceutically acceptable carrier thereof. The phrase “pharmaceutically acceptable” means the carrier, diluent, or excipient must be compatible with the other ingredients of the formulation and not deleterious to the recipient thereof.

Pharmaceutical compositions of the present technology can be used in the form of a solid, a solution, an emulsion, a dispersion, a patch, a micelle, a liposome, and the like, wherein the resulting composition contains one or more compounds of the present technology, as an active ingredient, in admixture with an organic or inorganic carrier or excipient suitable for enteral or parenteral applications. Compounds of the present disclosure may be combined, for example, with the usual non-toxic, pharmaceutically acceptable carriers for tablets, pellets, capsules, suppositories, solutions, emulsions, suspensions, and any other form suitable for use. The carriers which can be used include glucose, lactose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents and perfumes may be used. The compounds are included in the pharmaceutical composition in an amount sufficient to produce the desired effect upon the process or disease condition.

The pharmaceutical compositions may be in the form of a sterile injectable suspension. This suspension may be formulated according to known methods using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Sterile, fixed oils are conventionally employed as a solvent or suspending medium. For this purpose any bland fixed oil may be employed including synthetic mono- or diglycerides, fatty acids (including oleic acid), naturally occurring vegetable oils like sesame oil, coconut oil, peanut oil, cottonseed oil, etc., or synthetic fatty vehicles like ethyl oleate or the like. Buffers, preservatives, antioxidants, and the like can be incorporated as required.

The compounds of the technology may also be administered in the form of suppositories for rectal administration. These compositions may be prepared by mixing the compounds having formula (I) with a suitable non-irritating excipient, such as cocoa butter, synthetic glyceride esters of polyethylene glycols, which are solid at ordinary temperatures, but liquefy and/or dissolve in the rectal cavity to release the drug.

Pharmaceutical compositions containing compounds of the present disclosure may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of a sweetening agent such as sucrose, lactose, or saccharin, flavoring agents such as peppermint, oil of wintergreen or cherry, coloring agents and preserving agents in order to provide pharmaceutically elegant and palatable preparations. Tablets containing technology compounds in admixture with non-toxic pharmaceutically acceptable excipients may also be manufactured by known methods. The excipients used may be, for example, (1) inert diluents such as calcium carbonate, lactose, calcium phosphate or sodium phosphate; (2) granulating and disintegrating agents such as corn starch, potato starch or alginic acid; (3) binding agents such as gum tragacanth, corn starch, gelatin or acacia, and (4) lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

In some cases, formulations for oral use may be in the form of hard gelatin capsules wherein the compounds are mixed with an inert solid diluent, for example, calcium carbonate, calcium phosphate or kaolin. They may also be in the form of soft gelatin capsules wherein the technology compounds are mixed with water or an oil medium, for example, peanut oil, liquid paraffin or olive oil.

The compounds of the technology may also be administered as pharmaceutical compositions in a form suitable for topical use, for example, as oily suspensions, as solutions or suspensions in aqueous liquids or nonaqueous liquids, or as oil-in-water or water-in-oil liquid emulsions.

Pharmaceutical compositions may be prepared by combining a therapeutically effective amount of at least one compound according to the present technology, or a pharmaceutically acceptable salt thereof, as an active ingredient with conventional ophthalmically acceptable pharmaceutical excipients and by preparation of unit dosage suitable for topical ocular use. The therapeutically efficient amount typically is between about 0.001 and about 5% (w/v), preferably about 0.001 to about 2.0% (w/v) in liquid formulations.

For ophthalmic application, preferably solutions are prepared using a physiological saline solution as a major vehicle. The pH of such ophthalmic solutions should preferably be maintained between 4.5 and 8.0 with an appropriate buffer system, a neutral pH being preferred but not essential. The formulations may also contain conventional pharmaceutically acceptable preservatives, stabilizers and surfactants.

Preferred preservatives that may be used in the pharmaceutical compositions of the present technology include, but are not limited to, benzalkonium chloride, chlorobutanol, thimerosal, phenylmercuric acetate and phenylmercuric nitrate.

A preferred surfactant is, for example, Tween 80. Likewise, various preferred vehicles may be used in the ophthalmic preparations of the present technology. These vehicles include, but are not limited to, polyvinyl alcohol, povidone, hydroxypropyl methyl cellulose, poloxamers, carboxymethyl cellulose, hydroxyethyl cellulose cyclodextrin and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. Accordingly, buffers include acetate buffers, citrate buffers, phosphate buffers and borate buffers. Acids or bases may be used to adjust the pH of these formulations as needed.

In a similar manner, an ophthalmically acceptable antioxidant for use in the present technology includes, but is not limited to, sodium metabisulfite, sodium thiosulfate, acetylcysteine, butylated hydroxyanisole and butylated hydroxytoluene.

Other excipient components which may be included in the ophthalmic preparations are chelating agents. The preferred chelating agent is edentate disodium, although other chelating agents may also be used in place of or in conjunction with it.

The actual dose of the active compounds of the present technology depends on the specific compound, and on the condition to be treated; the selection of the appropriate dose is well within the knowledge of the skilled artisan.

The ophthalmic formulations of the present technology are conveniently packaged in forms suitable for metered application, such as in containers equipped with a dropper, to facilitate application to the eye. Containers suitable for dropwise application are usually made of suitable inert, non-toxic plastic material, and generally contain between about 0.5 and about 15 ml solution. One package may contain one or more unit doses. Especially preservative-free solutions are often formulated in non-resealable containers containing up to about ten, preferably up to about five units doses, where a typical unit dose is from one to about 8 drops, preferably one to about 3 drops. The volume of one drop usually is about 20-35 μl.

As will be evident to those skilled in the art, individual isomeric forms can be obtained by separation of mixtures thereof in conventional manner. For example, in the case of diastereoisomeric isomers, chromatographic separation may be employed.

The present technology also provides pharmaceutical kits for the treatment or prevention of cataract and presbyopia or cataract. The patient may be a human or animal patient. The kit comprises a specific amount of the individual doses in a package containing a pharmaceutically effective amount of at least one compound having formula (I). The kit can further include instructions for use of the kit. The specified amount of individual doses may contain from about 1 to about 100 individual dosages.

The presently described technology and its advantages will be better understood by reference to the following examples. These examples are provided to describe specific embodiments of the present technology. By providing these specific examples, it is not intended limit the scope and spirit of the present technology. It will be understood by those skilled in the art that the full scope of the presently described technology encompasses the subject matter defined by the claims appending this specification, and any alterations, modifications, or equivalents of those claims.

### EXAMPLES

### Example 1: Prevention of Aggregation of AAC by the Example Compound CAP4349 (Tafamids)

It was determined that CAP4349 protects hAAC from UV-induced aggregation in a concentration-dependent manner. The meglumine salt of Tafamidis (CAP4349-M) was tested for its ability to prevent hAAC aggregation using SDS/PAGE and absorbance.

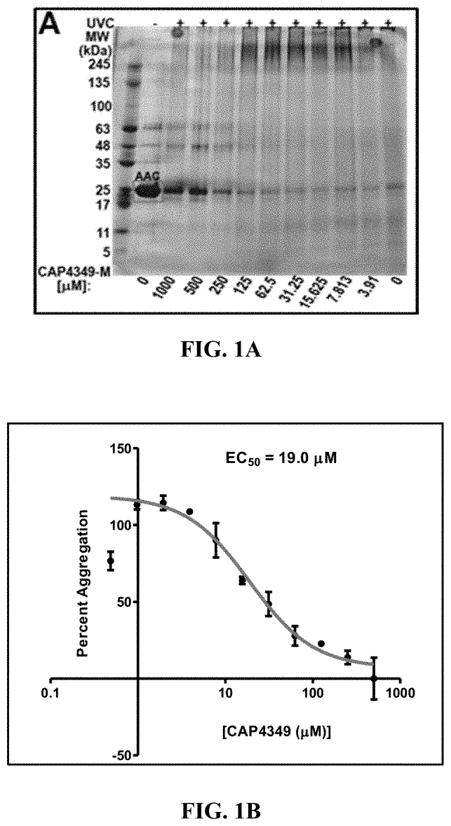

hAAC, with or without CAP4349-M (meglumine salt, 0-1000 μM), was exposed to UV light for 15 minutes and analyzed on a 4-20% gradient SDS-PAGE gel. CAP4349-M demonstrated a dose-dependent prevention of hAAC aggregation, which is apparent from the intensities of the bands of the aggregated and non-aggregated form on the SDS-PAGE gel (FIG. 1A).

In another study, hAAC, with or without CAP4349-M, was exposed to UV light for 60 minutes and the resulting aggregation was measured by absorbance. The results are shown in FIG. 1B in terms of mean percentage absorbance change±SD (n=3 or 4). The EC₅₀ value, which is the half-maximal response of hAAC aggregation, was determined to be19.7(±1.4) μM.

### Example 2: Example Compound CAP4349 Augments Chaperone-like Activity (CLA) of hAAC in a Dose-Dependent Manner

Post translational modification, due to UV light, oxidation, glycation, crosslinking, and proteolysis can affect the CLA of AAC and its ability to prevent the aggregation of target proteins. These modifications lead to aggregation of other lens crystallins, which culminate in the development of lens opacity and cataract development. Since CAP4349 prevents aggregation of hACC, the effect of CAP4349 on the CLA activity of AAC was evaluated using in vitro chaperone assays with (i) lysozyme as a model client protein, and (ii) bovine γ-crystallin (BGC), as a physiologically relevant lenticular protein.

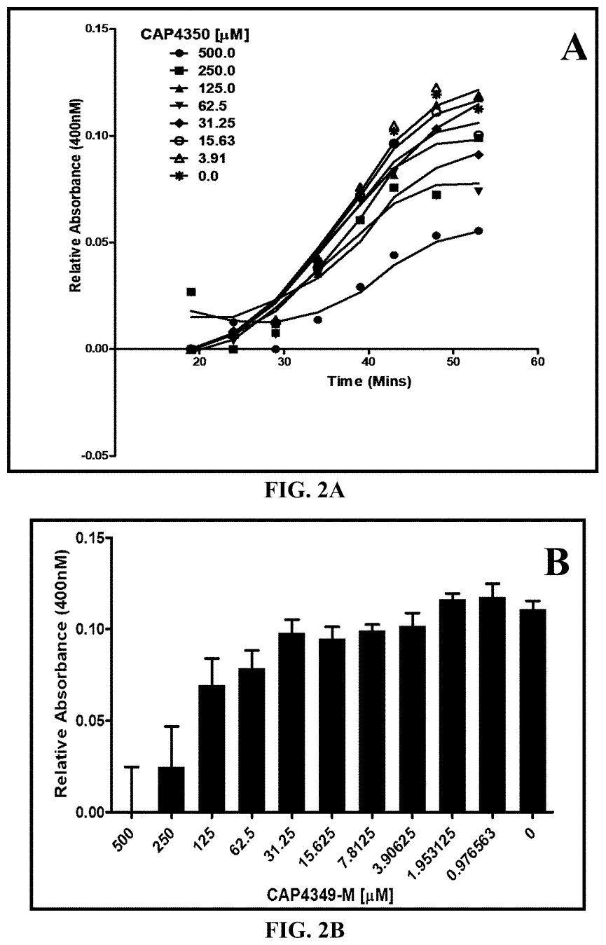

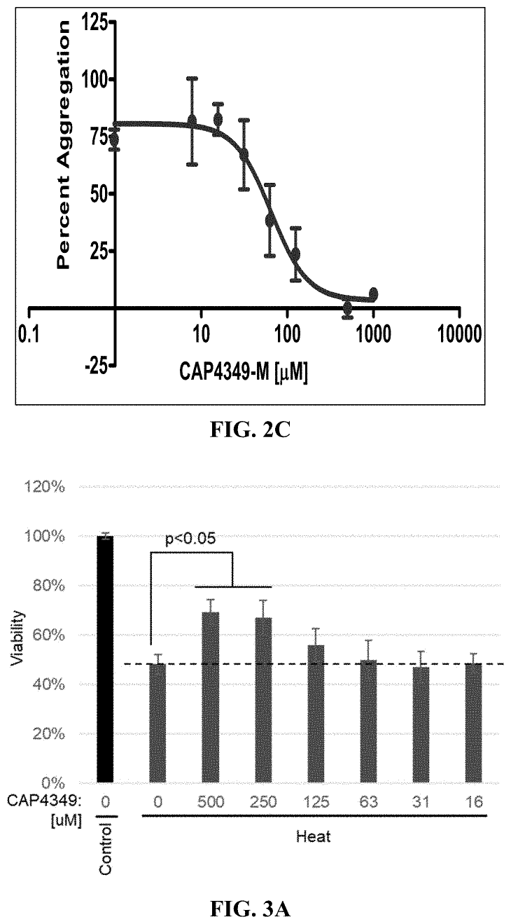

Heat-induced aggregation of lysozyme: In the presence of a reducing agent (DTT) and heat (37° C.), lysozyme undergoes denaturation and aggregation. The CLA activity of AAC can protect lysozyme against heat and DTT-induced aggregation (Robey, R. L. et al. (1997), Journal of Heterocyclic Chemistry, 34: (2)413-428). In order to examine the effect of CAP4349 on the ability of ACC to protect lysozyme against heat and DTT-induced aggregation, AAC (250 μμ/ml) was mixed with various concentrations of CAP4349-M and incubated for 1 hour at room temperature, followed by the addition of lysozyme (500 μg/ml . Aggregation reaction was initiated at 37° C. by the addition of 10 mM DTT. Aggregation was monitored as changes in light scattering via measurement of absorbance at 400 nm. It was found that in the presence of CAP4349, AAC demonstrated an increased protection of lysozyme from aggregation. The effect was dose dependent (FIG. 2A). These results suggest that CAP4349, upon interaction with AAC, increases its CLA.

UV-Induced Aggregation of BGC: Exposure to UV radiation is a contributing factor to cataract formation. Studies have shown that exposure of lenticular γ-crystallin (GC) to UV radiation in vitro leads to photo-aggregation and formation of HMW aggregates (Gilbert, Adam M. et al., (2007), Bioorganic & Medicinal Chemistry Letters, 17(5):1189-1192). AAC prevents the thermal and UV-induced aggregation of GC (Horowitz J. (1992), Proc. Nat. Acad. Sci. USA 89:10449-10453). Therefore, the effect of CAP4349 on the ability of AAC to prevent the aggregation of a physiologically relevant client protein, such as GC was examined. It was found that CAP4349 increased the protection provided by AAC against UV-induced BGC aggregation in a dose-dependent manner (FIG. 2B), suggesting that interaction of CAP4349 with AAC enhances its CLA towards the client protein, lenticular γ-crystallin.

Determination of efficacy of CAP4349 (EC₅₀) in enhancing CLA of ACC: AAC (250 μg/ml) was mixed with various concentration of CAP4349-M and incubated for 1 hour at room temperature followed by addition of purified BGC (500 μg/ml). The aggregation reaction was initiated by exposing the reaction mixture to UVB radiation. Aggregation was monitored for changes in light scattering by measuring absorbance at 600 nm at t=0, 15, 30 and 45 minutes. Mean percentage absorbance±SD from triplicate or quadruplicate wells plotted on a graph (FIG. 2C). EC₅₀ value was calculated as 64.8 (±1.3) ρM, which is the concentration of CAP4349-M concentration producing half-maximal response of BGC aggregation in presence of AAC.

### Example 3: Protection of Heat and UV Induced Cell Death of Human Lens Epithelial Cells by Example Compound CAP4349

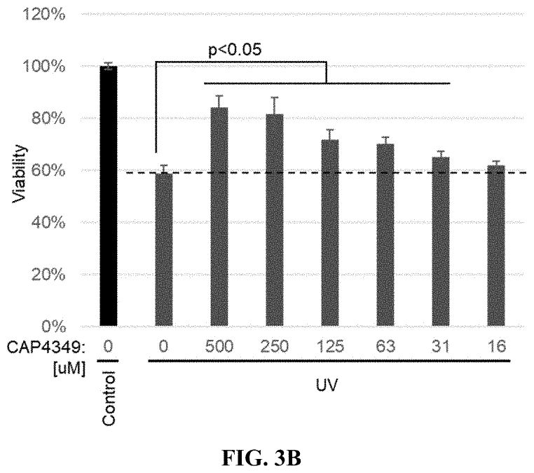

Efficacy of CAP4349 in protection of human lens epithelial cells from stress, as a measure of cellular AC function: Heating of intact human lenses dramatically promotes conversion of soluble AC into HMW aggregates and AC is responsible for protecting human lens epithelial (HLE) cells from thermal stress induced cell death (Peschek J et al. (2009), Proc. Natl. Acad. Sci. USA. 106(32):13272-13277). Survival of HLE cells following UV stress also depends on AC activity (Kumar P. et al., (2007) Biochem. J., 408:251-258). Assays for measuring cell-survival assays following heat or UV exposure were developed as a potential surrogate for AC chaperone activity. HLE cell line SRA 01/04 cells were pre-incubated for 2 hours with CAP4349 and then exposed to heat (50° C. for 40 m) or UV light (48 seconds). Results showed that CAP4349 protects the cells from heat-induced and UV-induced cell death in a dose-dependent manner (FIGS. 3A and 3B), suggesting that the increased CLA of AAC in the presence of CAP4349 is able to protect HLE cells from stress. CAP4349 does not alter the viability under normal conditions.

### Example 4: Example Compound CAP4349 Reduces Cellular Aggregates of AAC

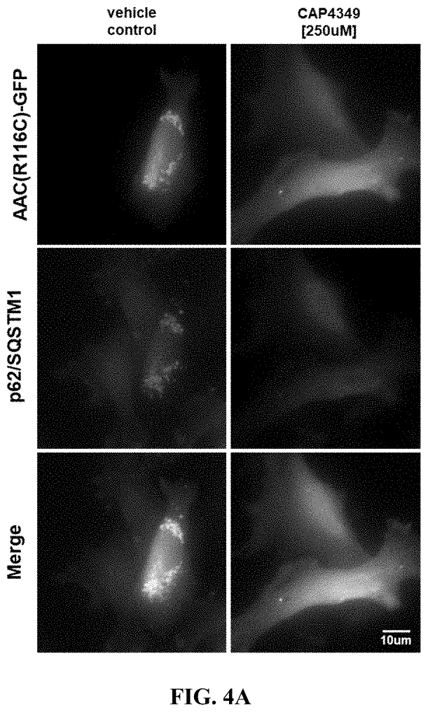

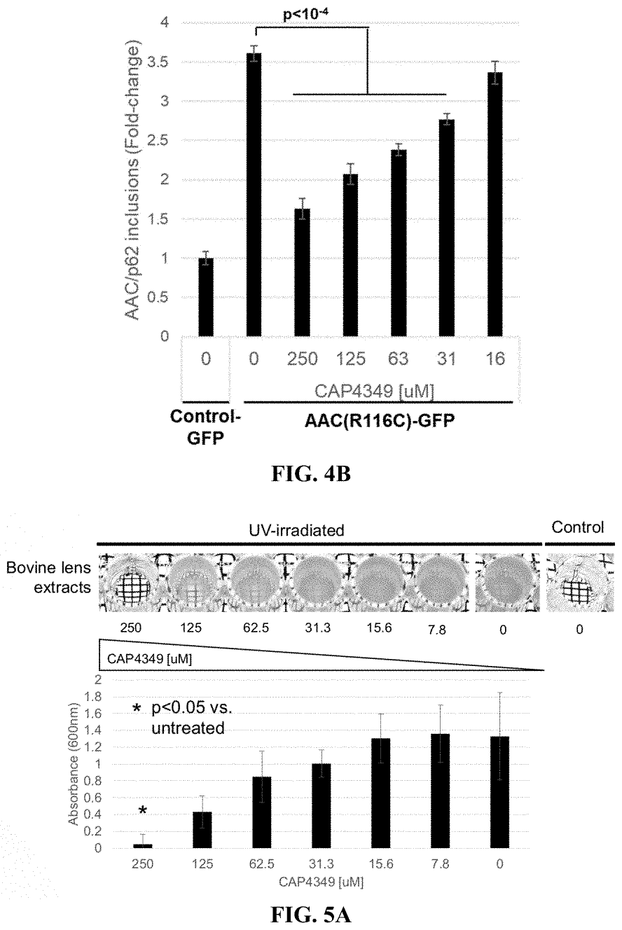

Cellular effects of CAP4349 were evaluated using an automated high-content assay for AAC aggregation. AAC(R116C)-GFP has been reported to form p62 positive inclusions (p62 is known to co-localize with ubiquitinated aggregates in several protein aggregation diseases) (Neal R, et al. (2010) Mol. Vis. 16:2137-2145.). Experiments were carried out to determine whether CAP4349 had any effect on co-localization of AAC and p62 in HeLa cells. Results showed that aggregates of AAC(R116C)-GFP in HeLa cells were co-localized with p62 and that these aggregates decreased upon treatment with CAP4349 (FIG. 4A). This reduction in aggregate formation was found to be statistically significant (FIG. 4B).

### Example 5: CAP4349 Prevents the Aggregation of Bovine and Human Lens Soluble Extracts when Exposed to UV Radiation

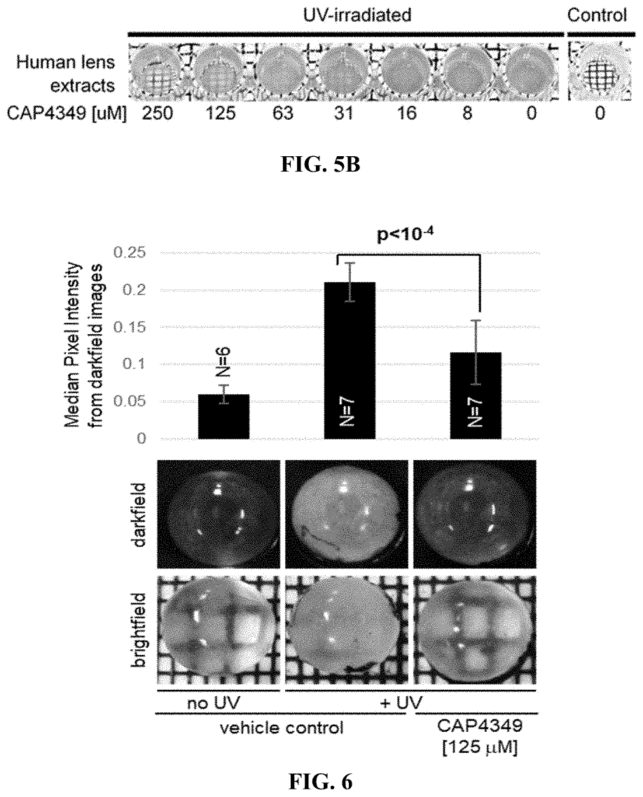

In the Examples above, CAP4349 was shown to protect recombinant hAAC from UV-induced aggregation and promotes its CLA on client proteins. Next, the ability of CAP4349 to prevent UV-induced opacification in a more complex and physiologically-relevant system was examined. Both bovine and human lens lysates contain AAC as well as multiple endogenous client proteins, all of which are present at physiological ratios in a physiologically-relevant milieu of ions, redox pairs and antioxidants. CAP4349 protected both bovine (FIG. 5A) and human (FIG. 5B) lysates from UV-induced opacification in a dose-dependent manner.

### Example 6: CAP4349 Increases Lens Clarity and Delays UV Induced Cataract Formation ex vivo

Porcine lenses were pretreated with 125 μM CAP4349 (or vehicle) and UV irradiated (or not). Porcine lenses (Sierra for Medical Science, Inc., Whittier, CA) have been used previously in ex vivo models of cataracts (Raju, M. (2014) Biochemistry, 53(16):2615-2623.). CAP4349 was found to prevent UV-induced opacification of porcine lenses ex vivo (FIG. 6). The ability of CAP4349 to protect whole intact lenses from opacification strongly suggests that the compound can do so in in vivo animal models of cataract.

### Example 7: Prodrugs of CAP4349

The maximum solubility for the meglumine salt of CAP4349 is 0.0323 mg/ml, (Drug bank: https://www.drugbank.ca/salts/DBSALT002673). Topical ophthalmic drugs generally exhibit bioavailability in 2-5% range (Gower NJD et al., (2016), BMC Ophthalmol. 16:11). Thus, the maximum concentration of meglumine formulated CAP4349 in the lens would be about 5 μM, which is below its EC₅₀ value (19.5 μM) for the prevention of hACC aggregation. Therefore, in order to improve the aqueous solubility and bioavailability, the present disclosure provides prodrugs of CAP4349.

One phosphate prodrug and one meglumine salt of CAP4349 were prepared, as examples, using Scheme 1 shown below. These compounds are referred to as CAP4357 and CAP4350 (meglumine salt of CAP4349), respectively.

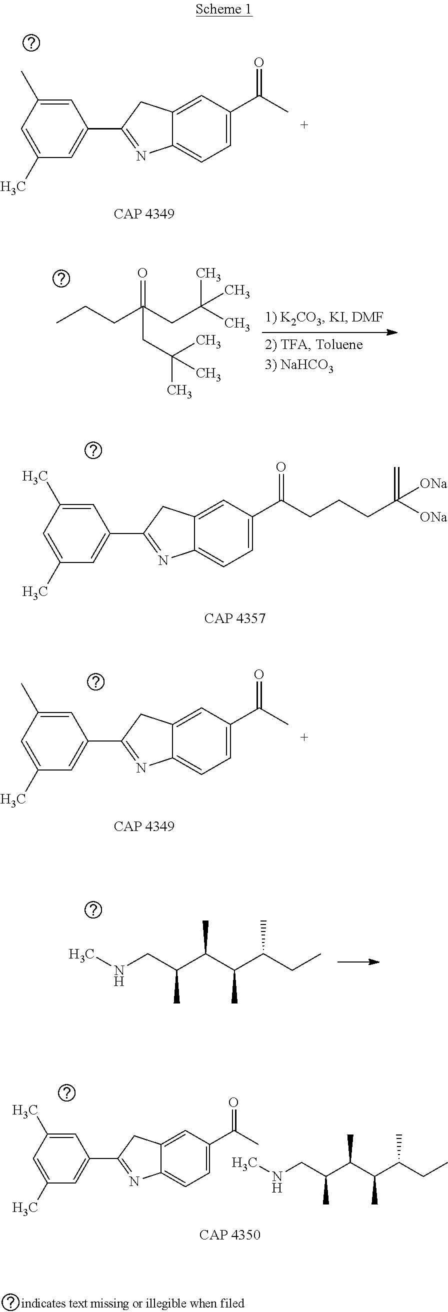

To a solution of Tafamidis, CAP4349 (308 mg) in DMF (10 mL) was added K2CO3 (220 mg) and KI (132 mg) followed by di-tert-butyl chloromethyl phosphate at room temperature while stirring. The reaction mixture was stirred at 70° C. for two hours. The reaction was quenched with ice and extracted three times in Ethyl Acetate. The combined organic layer was washed with water followed by 1% aqueous solution of sodium thiosulfate, and lastly with Brine, yielding the intermediate t-butyl protected phosphate. The t-butyl protected phosphate intermediate was converted to disodium salt in two steps by cleaving the protecting group with TFA followed by NaHCO₃ treatment in the presence of water and THF resulting in disodium salt of CAP4357, as a white solid. CAP4357 demonstrated an aqueous solubility of 25 mg/ml, which was increased to 40 mg/ml when formulated in 5% aqueous (2-Hydroxypropyl)-β-cyclodextrin (HPBCD)). This represents a 774- and 1,238-fold increase in aqueous and HPBCD solubility, respectively.

The acid group present in CAP4349 is amenable for the introduction of additional promoieties which can be converted to active drug by the enzymes present in the eye (Azema, Joelle et al., Bioorganic & Medicinal Chemistry, 14 (8), 2006, p. 2569-2580; Jerzy Golik et. al., Bioorg. Med. Chem. Lett. 6(15), 1996, p. 1837-1842; A. Mantyla et al., Tetrahedron Lett. 43, 2003, p. 3793-3794; and 53. Jeffrey P et al., J. Med. Chem., 42, (16), 1999, p. 3094-3100).

A general strategy for the design of prodrugs of CAP4349 is presented in Scheme 2.

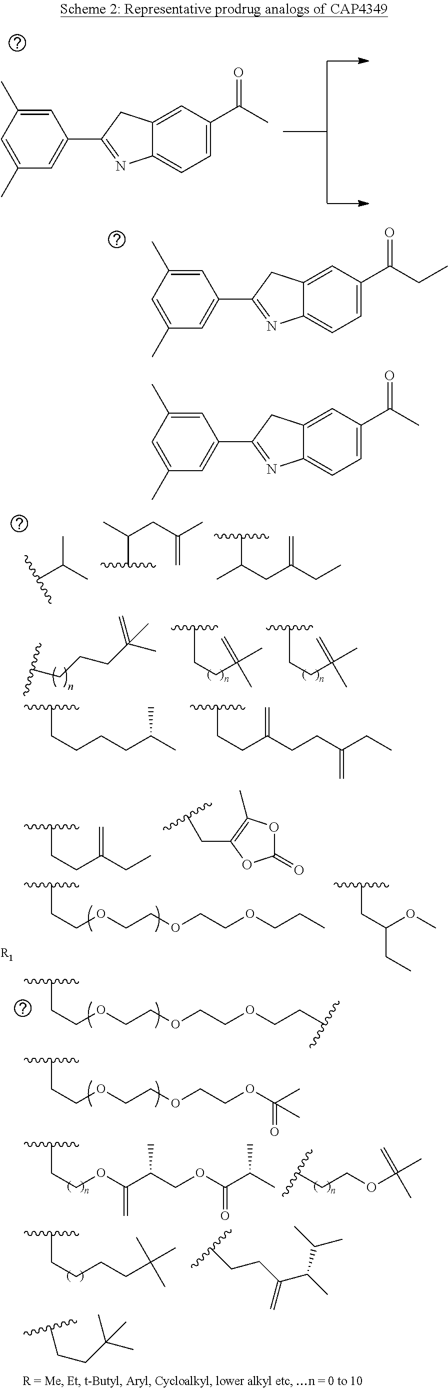

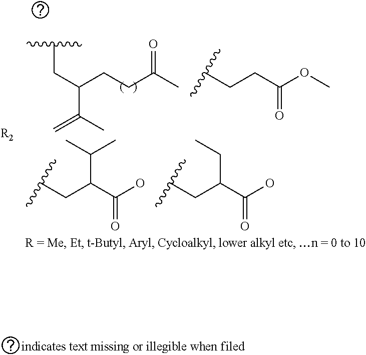

Several prodrugs were synthesized using the above scheme as shown below in Table 1.

TABLE 1
Characteristics of subset of prodrugs of CAP4349 designed and synthesized

| Cmpd. | Mol. | Mol. Wt. | Purity | Yield |
|---|---|---|---|---|
| ID | Str. | (Da) | (%) | (%) |
| CAP4354 | 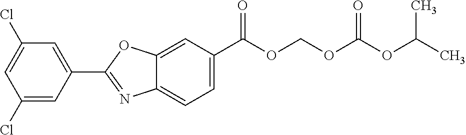 [SMILES: CC(C)OC(=O)OCOC(=O)c1ccc2nc(-c3cc(Cl)cc(Cl)c3)oc2c1] | 424.23 | 97 | 61 |
| CAP4355 | 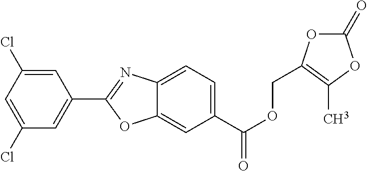 [SMILES: Cc1oc(=O)oc1COC(=O)c1ccc2nc(-c3cc(Cl)cc(Cl)c3)oc2c1] | 420.20 | 95 | 59 |
| CAP4356 | 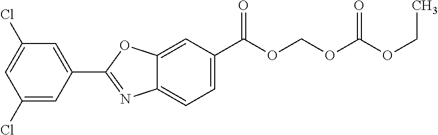 [SMILES: CCOC(=O)OCOC(=O)c1ccc2nc(-c3cc(Cl)cc(Cl)c3)oc2c1] | 410.20 | 97 | 60 |
| CAP4357 | 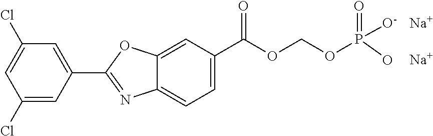 [SMILES: O=C(OCOP(=O)([O-])O)c1ccc2nc(-c3cc(Cl)cc(Cl)c3)oc2c1 ; [Na+] ; [Na+]] | 462.08 | 97 | 30 (3 steps) |
| CAP4358 | 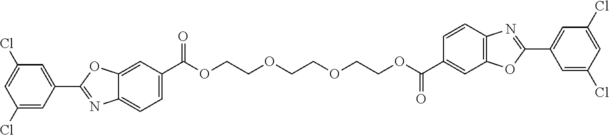 [SMILES: O=C(OCCOCCOCCOC(=O)c1ccc2nc(-c3cc(Cl)cc(Cl)c3)oc2c1)c1ccc2nc(-c3cc(Cl)cc(Cl)c3)oc2c1] | 730.37 | 96 | 45 |
| CAP4359 | 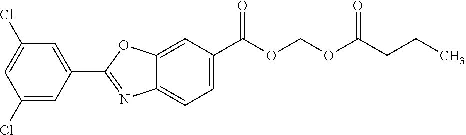 [SMILES: CCCC(=O)OCOC(=O)c1ccc2nc(-c3cc(Cl)cc(Cl)c3)oc2c1] | 408.23 | 97 | 60 |

Spectroscopic (NMR) data for the above compounds are provided in the following:

### CAP4354: 1H NMR (500 MHz, CDCl3): δ 8.32 (br s, 1H), 8.17-8.14 (m, 3H), 7.83 (d, J=10 Hz, 1H), 7.56 (br s, 1H), 6.05 (s, 2H), 2.64 (m, 1H), 1.22 (s, 6H).

### CAP4355: 1H NMR (500 MHz, CDCl3): δ 8.38 (br s, 1H), 8.24-8.23 (m, 3H), 7.83 (d, J=10 Hz, 1H), 7.56 (br s, 1H), 5.24 (s, 2H), 2.26 (s, 3H).

### CAP4356: 1H NMR (500 MHz, CDCl13):δ 8.32 (br s, 1H), 8.17-8.14 (m, 3H), 7.88 (d, J=8.5 Hz, 1H), 7.56 (br s, 1H), 6.05 (s, 2H), 4.29 (q, J=7.5 Hz, 2H), 1.34 (t, J=7 Hz, 3H).

### CAP4357: 1H NMR (500 MHz, DMSO): δ 8.38 (br s, 1H), 8.18 (d, J=5 Hz, 2H), 8.09 (d, J=10 Hz, 1H), 8.01 (d, J=10 Hz, 1H), 7.98 (br t, J=5 Hz, 1H), 4.45 (d, J=5 Hz, 2H).

### CAP4358: 1H NMR (500 MHz, CDCl3): δ 8.31 (br s, 2H), 8.09-8.07 (m, 6H), 7.72 (d, J=8.5 Hz, 2H), 7.52 (br s, 2H), 4.53-4.51 (m, 6H), 3.86-3.90 (m, 6H).

### CAP4359: 1H NMR (500 MHz, CDCl3): δ 8.32 (br s, 1H), 8.17-8.14 (m, 3H), 7.82 (d, J=8.5 Hz, 1H), 7.56 (br s, 1H), 6.05 (s, 2H), 2.39 (t, J=7Hz, 2H), 1.73-1.64 (m, 2H), 0.96 (t, J=6.5Hz, 3H).

### Example 8: Enzymatic Evaluation of Conversion of Prodrugs into Active Metabolite

A key step in prodrug design is the incorporation of an activation mechanism that ensures the conversion of the prodrug into the active species in an efficient and/or controlled manner to meet the therapeutic needs of a given the medical application. The in vitro bioactivation of the prodrugs is examined using recombinant human carboxylesterase and phosphatase enzyme. All metabolic stability experiments are performed in triplicate in 96-well plate format. Methods for performing these studies are known. For example, see U.S. Pat. Nos. 9,402,912 and 9,402,913, and US Patent Application Publications US2014/0256651, US2014/0256612, and US2014/0256660, the contents of each which are incorporated herein by reference in their entireties.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present technology, and without departing from the spirit and scope thereof, can make various changes and modifications of the technology to adapt it to various usages and conditions. Thus, other embodiments are also within the scope of the following claims.

What is claimed is:

1. A method for treating presbyopia or cataract in a subject in need thereof, the method comprising administering to the subject a pharmaceutical composition comprising a therapeutically effective amount a compound having formula (I) 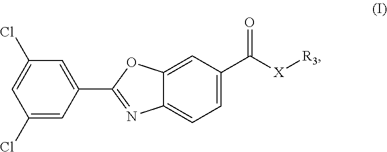
or a solvate or a pharmaceutically acceptable salt thereof,
wherein
R₃ is selected from the group consisting of hydrogen, an amino-acid, C₁₋₁₀ alkyl, C₁₋₁₀ branched-alkyl, C₁₋₁₀ hydroxyalkyl, C₁₋₁₀ haloalkyl, C₂₋₆ alkenyl, C₂-C₆ alkylaryl, C₁-C₆ alkyl (C₃-C₆) cycloalkyl, C₁₋₆ alkylNH, NC₁₋₆ dialkylamine, C_{1-6,} alkyl-pyrrolidine, C₁-C₆ alkyl-piperidine, C₁₋₁₀ alkyl morpholine, pthalidyl, 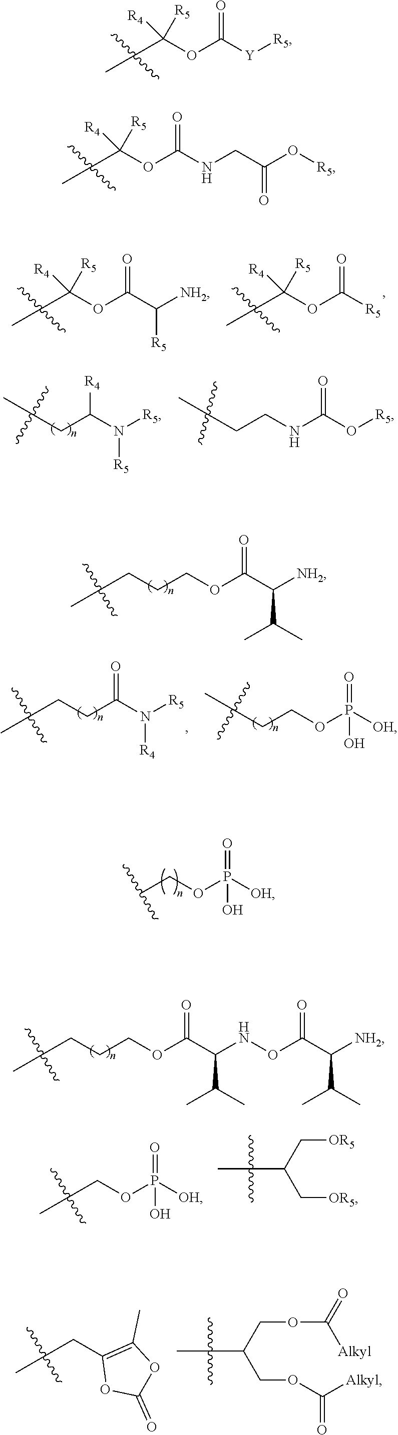 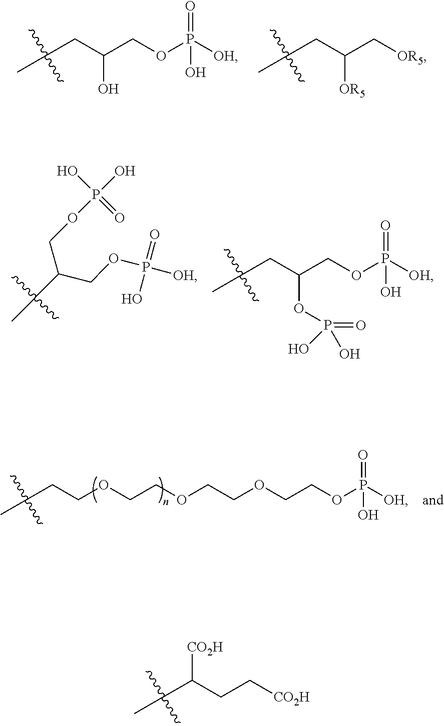
wherein n is a number between 0 and 6;
R₄ and R₅ are independently selected from hydrogen, C₁₋₆ alkyl, C₁₋₆ haloalkyl, C₁₋₆ hydroxyalkyl, C₂₋₆ alkoxyalkyl, aralkyl, C₂₋₆alkenyl, C₂₋₆ alkynyl, 3 to 6 membered cycloalkyl optionally substituted with at least one group selected from W, 4 to 6 membered heterocyclyl optionally substituted with at least one group selected from W,
wherein W is selected from C₁₋₆ alkyl, C₂₋₆ alkenyl, C₂₋₆ alkynyl, halo, NH₂, C₁₋₆ alkylNH, NC₁₋₆ dialkylamine, C₁₋₆ alkoxy, and hydroxyl; and Y is O, S, or NR₆, wherein R₆ is hydrogen or C₁₋₆ alkyl; and
wherein X is O or NR₆, wherein R₆ is hydrogen or C₁₋₆ alkyl.

2. The method of claim 1, wherein R₃ is an amino acid.

3. The method of claim 1, wherein R₄ is hydrogen and R₅ is a methyl or ethyl.

4. The method of claim 1, wherein R₃ is a C₁₋₆ alkyl.

5. The method of claim 1, wherein X is N.

6. The method of claim 1, wherein X is O.

7. The method of claim 1, wherein R₃ is 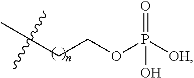
wherein n is 0.

8. The method of claim 1, wherein R₃ is 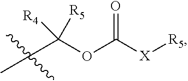
X is O, R₄ is H and R₅ is CH₃.

9. The method of claim 1, wherein R₃ is

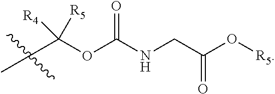

10. The method of claim 1, wherein the compound is 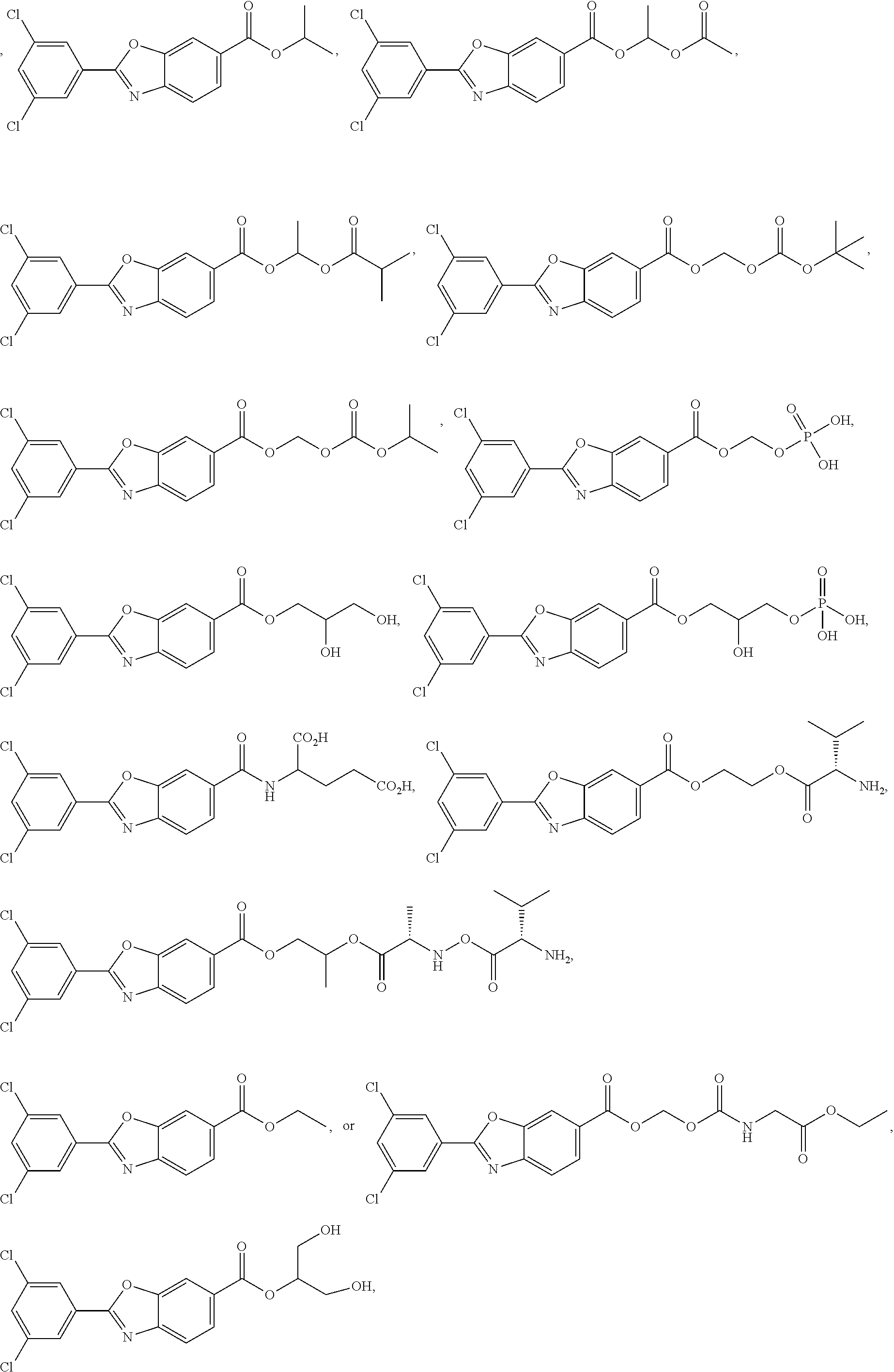
or a solvate or a pharmaceutically acceptable salt thereof.

11. The method of claim 1, wherein the subject is a human.

12. The method of claim 1, wherein the pharmaceutical composition is formulated for topical ophthalmic administration.

13. A method of preventing and/or treating transthyretin (TTR)-associated amyloidosis in a subject in need thereof, the method comprising administering to the subject a pharmaceutical composition comprising a therapeutically effective amount a compound having formula (I) 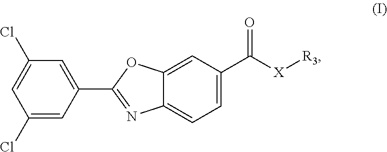
or a solvate or a pharmaceutically acceptable salt thereof,
wherein
R₃ is selected from the group consisting an amino-acid, C₁₋₁₀ alkyl, C₁₋₁₀ branched-alkyl, C₁₋₁₀ hydroxyalkyl, C₁₋₁₀ haloalkyl, C₂-C₆ alkenyl, C₂₋₆ alkylaryl, C₁₋₁₀ alkyl (C₃-C₆) cycloalkyl, C₁₋₆ alkylNH, NC₁₋₆ dialkylamine, C₁₋₆, alkyl-pyrrolidine, C₁-C₆ alkyl-piperidine, C₁₋₆ alkyl morpholine, pthalidyl, 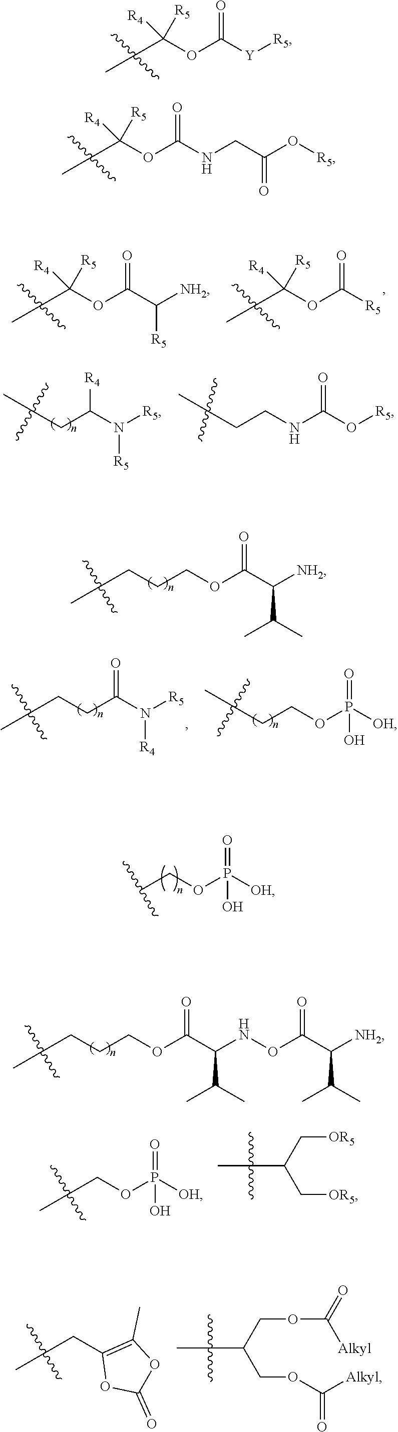 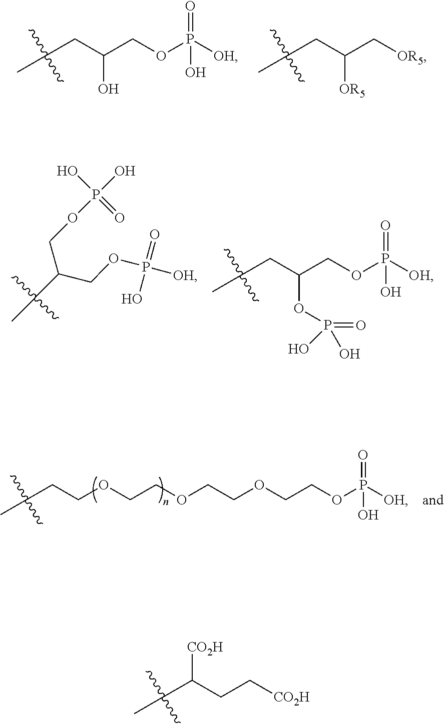
wherein n is a number between 0 and 6;
R₄ and R₅ are independently selected from hydrogen, C₁₋₆ alkyl, C₁₋₆ haloalkyl, C₁₋₆ hydroxyalkyl, C₂₋₆ alkoxyalkyl, aralkyl, C₂₋₆ alkenyl, C₂₋₆ alkynyl, 3 to 6 membered cycloalkyl optionally substituted with at least one group selected from W, 4 to 6 membered heterocyclyl optionally substituted with at least one group selected from W, wherein W is selected from C₁₋₆ alkyl, C₂₋₆ alkenyl, C₂₋₆ alkynyl, halo, NH₂, C₁₋₆ alkylNH, NC₁₋₆ dialkylamine, C₁₋₆ alkoxy, and hydroxyl; and Y is O, S, or NR₆, wherein R₆ is hydrogen or C₁₋₆ alkyl; and
wherein X is O or NR₆, wherein R₆ is hydrogen or C₁₋₆ alkyl.

14. The method of claim 13, wherein R₃ is an amino acid.

15. The method of claim 13, wherein R₄ is hydrogen and R₅ is a methyl or ethyl.

16. The method of claim 13, wherein R₃ is a C₁₋₆ alkyl.

17. The method of claim 13, wherein X is N.

18. The method of claim 13, wherein X is O.

19. The method of claim 13, wherein R₃ is 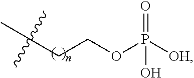
wherein n is 0.

20. The method of claim 13, wherein R₃ is 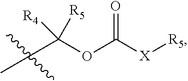
X is O, R₄ is H and R₅ is CH₃.

21. The method of claim 13, wherein R₃ is

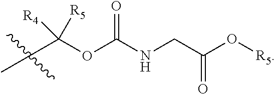

22. The method of claim 13, wherein the compound is 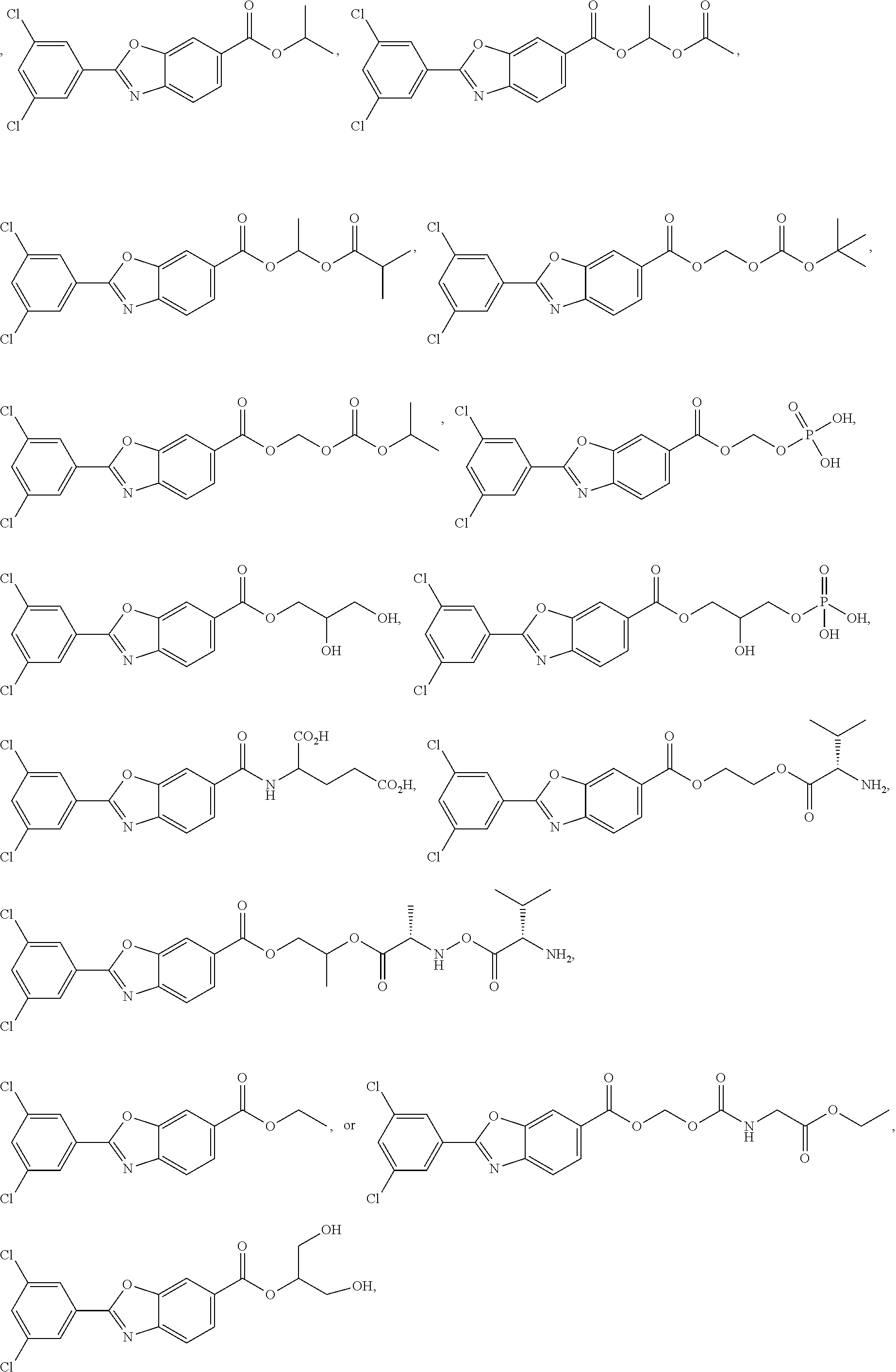
or a solvate or a pharmaceutically acceptable salt thereof.

23. The method of claim 13, wherein the subject is a human.

24. The method of claim 13, wherein the pharmaceutical composition is formulated for topical ophthalmic administration
